(19) United States
(12) Patent Application Publication
(10) Pub. No.: US 2023/0310745 A1
(43) Pub. Date: Oct. 5, 2023

(54) METHODS OF INTRAVENOUSLY ADMINISTERING DOFETILIDE

(71) Applicant: AltaThera Pharmaceuticals LLC, Chicago, IL (US)

(72) Inventor: Brandon Ira Kashfian, Chicago, IL (US)

(21) Appl. No.: 18/315,790

(22) Filed: May 11, 2023

Related U.S. Application Data
(63) Continuation-in-part of application No. 18/306,660, filed on Apr. 25, 2023.
Application No. 18/306,660 is a continuation-in-part of application No. 17/892,301, filed on Aug. 22, 2022.
Application No. 17/892,301 is a continuation-in-part of application No. 17/566,840, filed on Dec. 31, 2021, now Pat. No. 11,610,660.
(60) Provisional application No. 63/345,068, filed on May 24, 2022.
(60) Provisional application No. 63/345,068, filed on May 24, 2022.
(60) Provisional application No. 63/344,154, filed on May 20, 2022.
(60) Provisional application No. 63/344,154, filed on May 20, 2022.
(60) Provisional application No. 63/340,581, filed on May 11, 2022.
(60) Provisional application No. 63/340,581, filed on May 11, 2022.
(60) Provisional application No. 63/334,267, filed on Apr. 25, 2022.
(60) Provisional application No. 63/331,905, filed on Apr. 18, 2022.
(60) Provisional application No. 63/276,947, filed on Nov. 8, 2021.
(60) Provisional application No. 63/276,947, filed on Nov. 8, 2021.
(60) Provisional application No. 63/235,500, filed on Aug. 20, 2021.
(60) Provisional application No. 63/235,500, filed on Aug. 20, 2021.

(51) Int. Cl. A61M 5/172; G16H 20/17; A61K 31/145; A61K 9/00

(52) U.S. Cl. CPC A61M 5/1723; A61K 9/0019; A61K 31/145; G16H 20/17

(57) ABSTRACT

Methods of administering dofetilide in an amount effective for treating a cardiovascular condition are described. An initial IV loading dose can be administered over a period of about one hour. The patient can be discharged from the medical facility providing cardiac monitoring prior to administration of oral maintenance doses. The IV and oral doses can be administered in a manner such that maximum serum concentration of dofetilide is reached within about 1 hour of the start of the administration of the IV loading dose.

### CROSS-REFERENCE TO RELATED APPLICATIONS

This application is a Continuation-in-Part (CIP) application of U.S. Application No. 18/306,660 filed Apr. 25, 2023, which is a CIP application of U.S. Application No. 17/892,301 filed Aug. 22, 2022. The ‘301 Application is a CIP application of U.S. Application No. 17/566,840 filed Dec. 31, 2021, which issued as U.S. Pat. No. 11,610,660 on Mar. 21, 2023, and which application relies on the disclosure of and claims priority to and the benefit of the filing date of each of U.S. Provisional Application Nos. 63/235,500 filed Aug. 20, 2021 and 63/276,947 filed Nov. 8, 2021. The ‘301 Application relies on the disclosure of and claims priority to and the benefit of the filing date of each of U.S. Provisional Application Nos. 63/235,500 filed Aug. 20, 2021, 63/276,947 filed Nov. 8, 2021, 63/331,905 filed Apr. 18, 2022, 63/334,267 filed Apr. 25, 2022, 63/340,581 filed May 11, 2022, 63/344,154 filed May 20, 2022, and 63/345,068 filed May 24, 2022. The present application relies on the disclosure of and claims priority to and the benefit of the filing date of each of U.S. Provisional Application Nos. 63/340,581 filed May 11, 2022, 63/344,154 filed May 20, 2022, and 63/345,068 filed May 24, 2022. The disclosures of these applications are hereby incorporated by reference herein in their entireties.

### FIELD OF THE INVENTION

The present disclosure is directed to the field of cardiovascular pharmaceutics, more particularly compositions and methods for the treatment of cardiovascular conditions such as arrhythmias with intravenous anti-arrhythmics, such as dofetilide.

### BACKGROUND

Dofetilide is a class III antiarrhythmic agent which acts through blocking cardiac ion channels of the rapid component of the delayed rectifier potassium current Ikr. The agent, a sulfonamide, is approved to treat atrial fibrillation and atrial flutter. Dofetilide normalizes sinus rhythm by prolonging cardiac action potential duration and effective refractory period due to delayed repolarization without affecting conduction velocity. Dofetilide is currently approved in the US for oral administration under the brand name TIKOSYN® (Pfizer Inc., New York, NY). It is not approved for parenteral administration.

### SUMMARY OF THE INVENTION

Described herein are methods of administering dofetilide to a subject, such as a patient in need thereof, in an amount effective for treating a cardiovascular condition of the patient. Dofetilide can be administered to a subject who is currently in sinus rhythm, who has been recently cardioverted to sinus rhythm, or to convert the subject to sinus rhythm. In embodiments, the effective amount of dofetilide can be administered intravenously as a loading dose of about 0.1 to 12 µg/kg bodyweight over a duration of up to about 60 minutes. In embodiments, one or more maintenance dose(s) is/are given orally and/or infused intravenously as an IV maintenance dose of about 0.1 to 10 µg/kg/hr over a duration of at least 1 hour.

In embodiments, the cardiovascular condition is selected from atrial fibrillation, atrial flutter, atrial tachycardia, ventricular tachycardia, hemodynamically stable or unstable ventricular tachycardia, paroxysmal atrial fibrillation, ventricular fibrillation, ventricular arrhythmia, premature ventricular contractions (PVCs), paroxysmal supraventricular tachycardia, supraventricular tachycardia, junctional ectopic tachycardia, junctional tachycardia, heart failure, coronary artery disease, and pulmonary artery hypertension. The subject can be currently in sinus rhythm or recently cardioverted to sinus rhythm. Additionally, the dofetilide treatment can be used to convert the subject to sinus rhythm, such as for a subject with atrial fibrillation or atrial flutter or ventricular arrhythmia. The method can further include measuring a QT or QTc interval of the patient before, during, and/or after the administering, and selecting or adjusting the effective amount, the loading dose and/or the maintenance dose based on the QT or QTc interval at any point during administering the protocol. The method can further include measuring a creatinine clearance of the patient before the administering, and selecting or adjusting the effective amount, the loading dose and/or the maintenance dose based on the creatinine clearance.

Aspects of embodiments of the invention include Aspect 1, which is a method of intravenously administering dofetilide, comprising: administering an IV loading dosage of dofetilide to a subject over a period of about 1 hour in the amount in the range of ± 50% of a target oral dose; wherein the IV dosage is administered while the subject is in a facility capable of providing cardiac resuscitation and electrocardiographic monitoring.

Aspect 2 is a method of administering dofetilide, comprising: administering an IV loading dosage of dofetilide to a subject over a period of about 1 hour in an amount of about 62.5-750 µg; wherein the IV dosage is administered while the subject is in a facility capable of providing cardiac resuscitation and electrocardiographic monitoring; and after administering the IV dosage, and after discharging the subject from the facility capable of providing cardiac resuscitation and electrocardiographic monitoring, administering an oral dosage of dofetilide to the subject in an amount of 125 µg, 250 µg or 500 µg.

Aspect 3 is the method of Aspect 1 or 2, further comprising administering or optionally repeating oral administration of dofetilide at least once at a selected interval.

Aspect 4 is the method of Aspect 3, wherein the administering or repeating of the oral administration is performed after the subject is discharged from the facility capable of providing cardiac resuscitation and electrocardiographic monitoring.

Aspect 5 is the method of any of Aspects 1-4, wherein the subject has a cardiovascular condition selected from atrial fibrillation, atrial flutter, atrial tachycardia, ventricular tachycardia, hemodynamically stable or unstable ventricular tachycardia, paroxysmal atrial fibrillation, ventricular fibrillation, ventricular arrhythmia, premature ventricular contractions (PVCs), paroxysmal supraventricular tachycardia, supraventricular tachycardia, junctional ectopic tachycardia, junctional tachycardia, heart failure, coronary artery disease, and pulmonary artery hypertension.

Aspect 6 is the method of any of Aspects 1-5, wherein the subject is currently in sinus rhythm and/or has experienced cardioversion to sinus rhythm, or the treatment can be administered to a subject who is not in sinus rhythm, but to convert the subject to sinus rhythm.

Aspect 7 is the method of any of Aspects 1-6, wherein: the IV dosage is administered in an amount of: (a) about 62.5-187.5 µg; (b) about 125-375 µg; or (c) about 250-750 µg.

Aspect 8 is the method of any of Aspects 1-7, wherein: the target oral dose, the oral dosage and/or the repeated oral administration is: (a) 125 µg; or (b) 250 µg; or (c) 500 µg.

Aspect 9 is the method of any of Aspects 1-8, wherein the IV dosage is administered in an amount of: (a) about 62.5-137.5 µg, such as for when the subject is being initiated for an oral dose of dofetilide in the amount of 125 µg; (b) about 125-275 µg, such as for when the subject is being initiated for or escalated to an oral dose of dofetilide in the amount of 250 µg; or (c) about 250-550 µg, such as for when the subject is being initiated for or escalated to an oral dose of dofetilide in the amount of 500 µg.

Aspect 10 is the method of any of Aspects 1-9, wherein the oral dosage is administered to the subject starting at least about 1 hour or 2 hours after completion of the IV dosage.

Aspect 11 is the method of any of Aspects 1-10, where in the IV dosage is administered as a single IV dosage or by way of several IV doses.

Aspect 12 is the method of any of Aspects 1-11, where in the IV dosage is administered in an amount that is up to 50% higher than the target oral dose or the oral dosage amount, up to 50% lower than the target oral dose or the oral dosage amount, in the range of ±30% of the target oral dose or the oral dosage amount, in the range of ±25% of the target oral dose or the oral dosage amount, in the range of ±20% of the target oral dose or the oral dosage amount, or in the range of ±10% of the target oral dose or the oral dosage amount.

Aspect 13 is the method of any of Aspects 1-12, wherein: the subject is being initiated or escalated to a higher dose of oral dofetilide; the administering of the IV loading dosage comprises: determining kidney function, such as a creatinine clearance, of the subject; determining a QT or QTc interval of the subject; administering to the subject an IV loading dosage of dofetilide, wherein the IV loading dosage is selected from an amount ranging from about 0.1-12 µg/kg, and determining a second QT or QTc interval of the subject.

Aspect 14 is the method of Aspect 13, wherein: if one or more oral dose(s) of dofetilide are administered to the subject, determining one or more subsequent QT or QTc interval of the subject between one or more of the oral doses; and administering one or more subsequent oral doses to the subject wherein the oral dose administered is selected from 125 µg, 250 µg, or 500 µg.

Aspect 15 is the method of any of Aspects 1-14, wherein the administering of the oral dose(s) begins about 1-12 hours after completion of the administering of the IV loading dose, such as beginning about 2-4 hours after, or about 4-6 hours after, or about 6-8 hours after, or about 8-10 hours after, or about 10-12 hours after.

Aspect 16 is the method of any of Aspects 1-15, wherein the administering of the oral dose(s) occurs at a 12-hour, 24-hour or 48-hour interval from a previous oral dose.

Aspect 17 is the method of any of Aspects 1-16, wherein a second oral dose of dofetilide is administered to the subject about 12-48 hours after the first oral dose.

Aspect 18 is the method of any of Aspects 1-17, wherein the IV loading or maintenance dose is administered in an amount of up to 750 µg, or up to 550 µg, or up to 300 µg, or up to 150 µg.

Aspect 19 is the method of any of Aspects 1-18, wherein the subject had received a prior dose of dofetilide prior to administration of the IV loading dose, such as about 12-24 hours prior to administration of the IV loading dose.

Aspect 20 is the method of any of Aspects 1-19, wherein the subject is capable of experiencing a dofetilide Cₘₐₓ steady state within about 2 hours of administration of the IV loading dose, such as within about 1.5 hours, or within about 1 hour, or within about 30 minutes.

Aspect 21 is the method of any of Aspects 1-20, wherein the subject has a creatinine clearance of >60 mL/min before being administered the IV dofetilide.

Aspect 22 is the method of any of Aspects 1-20, wherein the subject has a creatinine clearance of 40 mL/min to 60 mL/min.

Aspect 23 is the method of any of Aspects 1-20, wherein the subject has a creatinine clearance of 20 mL/min to <40 mL/min.

Aspect 24 is the method of any of Aspects 1-23, wherein the subject is discharged with instructions to self-administer subsequent oral doses of dofetilide every 12 to 48 hours.

Aspect 25 is the method of any of Aspects 1-24, further comprising measuring a QT or QTc interval of the subject before administration of the IV loading dose.

Aspect 26 is the method of any of Aspects 1-25, further comprising measuring a QT or QTc interval of the subject after administration of the IV loading dose and if an oral dose is administered, then before the first oral dose.

Aspect 27 is the method of any of Aspects 1-26, further comprising measuring a QT or QTc interval of the subject after administration of the second oral dose, if any, or any subsequent oral dose, if any.

Aspect 28 is the method of any of Aspects 1-27, wherein the amount of the second oral dose, if any, is different from the amount of the first oral dose, if any, such as the second oral dose being a lower amount, or the second oral dose being a higher amount.

Aspect 29 is the method of any of Aspects 1-28, wherein the subject is being treated for atrial fibrillation and/or atrial flutter, and optionally is in sinus rhythm, or is not in sinus rhythm but is being administered dofetilide for the purpose of conversion to sinus rhythm, or is in sinus rhythm due to having been converted to sinus rhythm with dofetilide, or another antiarrhythmic, or by cardioversion techniques.

Aspect 30 is the method of any of Aspects 1-29, wherein the IV loading dose is administered to the subject in an amount and over a period of time such that the dofetilide hydrochloride reaches or is capable of reaching a Cₘₐₓ in the subject that is at least about 70% of a steady state Cₘₐₓ for an oral dosing protocol of 125 µg, 250 µg, or 500 µg dofetilide, such as about 75%, 80%, 85%, 87%, 90%, 92%, 95%, 97% or 99%.

Aspect 31 is a method of administering dofetilide, comprising: before administering an IV dosage of dofetilide to a subject, determining a creatinine clearance (CrCl) of the subject, and selecting an amount of dofetilide to administer to the subject based on the creatinine clearance; and administering to the subject the IV dosage comprising the selected amount of dofetilide based on the creatinine clearance.

Aspect 32 is the method of Aspect 31, wherein the IV dosage is administered to the subject in a facility capable of providing cardiac resuscitation and continuous electrocardiographic monitoring.

Aspect 33 is the method of Aspect 31 or 32, further comprising administering one or more oral dosages of dofetilide to the subject out of the facility capable of providing cardiac resuscitation and continuous electrocardiographic monitoring.

Aspect 34 is the method of any of the Aspects herein, further comprising administering one or more IV maintenance dose of dofetilide after administering the IV dosage of dofetilide.

Aspect 35 is the method of any of the Aspects herein, further comprising administering one or more oral dose of dofetilide before or after administering the IV dosage of dofetilide.

Aspect 36 is the method of any of the Aspects herein, wherein: the IV dosage is administered over a period in the range of 1-5 hours.

Aspect 37 is the method of any of the Aspects herein, wherein: the IV dosage comprising the selected amount of dofetilide based on the creatinine clearance of the subject comprises an amount of dofetilide as follows:

| Maintenance Dose | Intravenous Dosage of Dofetilide | | |
|---|---|---|---|
| | Subject’s CrCl >60 mL/min | Subject’s CrCl 40-60 mL/min | Subject’s CrCl 20 to <40 mL/min |
| 125 µg | 50-150 µg | 100-250 µg | up to 125 µg |
| 250 µg | 100-350 µg | 200-500 µg | up to 250 µg |
| 500 µg | 300-800 µg | 400-1000 µg | up to 500 µg |

Aspect 38 is the method of any of the Aspects herein, wherein the subject is capable of experiencing a dofetilide Cₘₐₓ steady state within about 2 hours of administration of the IV dosage.

Aspect 39 is the method of any of the Aspects herein, wherein the IV dosage of dofetilide is administered: in a first higher amount for the subject with a CrCl of 20 mL/min to <40 mL/min; and in a second lower amount for the subject with a CrCl of 40 mL/min or greater.

Aspect 40 is the method of any of the Aspects herein, wherein the subject is discharged with instructions or a prescription to administer one or more oral doses of dofetilide at a 12 to 48 hour interval for a selected period of time.

Aspect 41 is the method of any of the Aspects herein, wherein the amount of dofetilide in one or more of the oral doses is different than the amount of dofetilide in one or more other of the oral doses.

Aspect 42 is the method of any of the Aspects herein, wherein the IV dosage is administered to the subject in an amount and over a period of time such that the dofetilide hydrochloride reaches or is capable of reaching a Cₘₐₓ in the subject that is at least about 70% of a steady state Cₘₐₓ for an oral dosing protocol of 125 µg, 250 µg, or 500 µg dofetilide.

Aspect 43 is the method of any of the Aspects herein, further comprising: obtaining a QT or QTc interval of the subject before administration of the IV dosage; and obtaining a QT or QTc interval of the subject during or after administration of the IV dosage, but before administration of any oral dosage; wherein the QT or QTc interval during or after administering the IV dosage is less than a 15% increase from the QT or QTc interval before administering the IV dosage.

### DETAILED DESCRIPTION OF VARIOUS EMBODIMENTS OF THE INVENTION

Reference will now be made in detail to various illustrative implementations. It is to be understood that the following discussion of the implementations is not intended to be limiting.

IV is intravenous.

PO means “per os” and refers to an oral dosing regimen.

BID means “bis in die” and means twice a day.

QD means “quaque die” and means once a day.

QID means “quater in die” and means four times a day.

Patient (or subject) refers to a human subject or patient.

BP is blood pressure.

HR is heart rate.

Renally impaired refers to patients/subjects having creatinine clearance rates of ≤60 mL/min, such as ≤30 mL/min.

Cₘₐₓ ss is the maximal concentration obtained at steady state.

QT is the interval measured from the start of the Q wave or the QRS complex, to the end of the T wave, where the Q wave corresponds to the beginning of ventricular depolarization and the T wave end corresponds to the end of ventricular repolarization.

QTc is the calculated interval that represents the QT interval corrected for heart rate and can be derived by mathematical correlation of the QT interval and the heart rate.

ΔQT is the difference between a QT measurement taken prior to the start of treatment and a QT measured after the start of treatment (e.g., during loading or maintenance).

ΔQTc is the difference between a QTc measurement taken prior to the start of treatment and a QTc measured after the start of treatment (e.g., during loading or maintenance).

The terms “treat,” “treating,” and “treatment” refer to any indicia of success in the treatment or amelioration of an injury, disease, or condition, including any objective or subjective parameter such as abatement; remission; diminishing of symptoms or making the injury disease, or condition more tolerable to the subject; slowing in the rate of degeneration or decline; or improving a subject’s physical or mental well-being. The treatment or amelioration of symptoms can be based on objective or subjective parameters, including the results of a physical examination, neuropsychiatric examinations, or psychiatric evaluation.

Hospital refers to a medical facility staffed and equipped to provide continuous ECG monitoring and cardiac resuscitation to patients, if needed. Typically, the medical personnel are trained in the management of serious ventricular arrhythmias.

Escalation means increasing the dofetilide dosage of a patient already receiving dofetilide, for example where a subject is currently taking a specific amount of an oral dose and escalation involves administering one or more IV doses to escalate the subject to a higher target oral dose.

The term “about” used herein in the context of quantitative measurements means the indicated amount ±10%. For example, “about 2 mg” can mean 1.8-2.2 mg.

### IV and Oral Dosing

According to embodiments of the invention, dofetilide can be administered to a subject with a cardiovascular condition, such as a cardiovascular condition selected from atrial fibrillation, atrial flutter, atrial tachycardia, ventricular tachycardia, hemodynamically stable or unstable ventricular tachycardia, paroxysmal atrial fibrillation, ventricular fibrillation, ventricular arrhythmia, premature ventricular contractions (PVCs), paroxysmal supraventricular tachycardia, supraventricular tachycardia, junctional ectopic tachycardia, junctional tachycardia, heart failure, coronary artery disease, and pulmonary artery hypertension.

The subject can be currently in sinus rhythm or recently cardioverted to sinus rhythm. Additionally, the dofetilide treatment can be used to convert the subject to sinus rhythm, such as for a subject with atrial fibrillation or atrial flutter or ventricular arrhythmia.

The dofetilide can be administered intravenously as a loading dose in the range of 0.1 to 8 µg/kg, including 0.1, 0.2, 0.3, 0.4, 0.5, 0.6, 0.7, 0.8, 0.9, 1.0, 1.2, 1.4, 1.5, 1.6, 1.8, 2.0, 2.2, 2.4, 2.5, 2.6, 2.8, 3.0, 3.2, 3.4, 3.5, 3.6, 3.8, 4.0, 4.2, 4.4, 4.5, 4.6, 4.8, 5.0, 5.2, 5.4, 5.5, 5.6, 5.8, 6.0, 6.2, 6.4, 6.5, 6.6, 6.8, 7.0, 7.2, 7.4, 7.5, 7.6, 7.8, 8.0, µg/kg, or in any range with any of these values as lower or upper values, such as 1.5 to 4.4 µg/kg, 3.2 to 6.8 µg/kg, 2.6 to 5.4 µg/kg, 2.0 to 6.0 µg/kg, 1.8 to 4.8 µg/kg, and so on. Higher loading doses, such as those exceeding 8.0 µg/kg, such as 8.2 to 12.0 µg/kg, including 8.2, 8.4, 8.5, 8.6, 8.8, 9.0, 9.2, 9.4, 9.5, 9.6, 9.8, 10.0, 10.2, 10.4, 10.5, 10.6, 10.8, 11.0, 11.2, 11.4, 11.5, 11.6, 11.8, and 12.0 µg/kg, or in any range with any of these values as lower and upper values, such as 8.2 to 10 µg/kg may be possible, such as, if administered over longer durations.

In embodiments, the loading dose amount is selected from an amount in the range of ±50% of a target oral dose or a target maintenance dose, such as in the range of ±5%, ±10%, ±15%, ±20%, ±25%, ±30%, ±35%, ±40%, or ±45%.

In embodiments, for a target oral dose of 500 µg, the loading dose amount may be in the range of about 250 µg to about 1000 µg, such as about 275 µg to about 750 µg, about 300 µg to about 725 µg, about 325 µg to about 700 µg, about 350 µg to about 650 µg, about 375 µg to about 625 µg, about 400 µg to about 600 µg, about 425 µg to about 575 µg, about 450 µg to about 550 µg, or about 475 µg to about 525 µg.

In embodiments, for a target oral dose of 250 µg, the loading dose may be in the range of about 125 µg to about 375 µg, such as about 137.5 µg to about 362.5 µg, about 150 µg to about 350 µg, about 162.5 to about 337.5 µg, about 175 µg to about 325 µg, about 187.5 µg to about 312.5 µg, about 200 µg to about 300 µg, about 212.5 µg to about 287.5 µg, about 225 µg to about 275 µg, or about 237.5 µg to about 262.5 µg.

In embodiments, for a target oral dose of 125 µg, the loading dose may be in the range of about 62.5 µg to about 187.5 µg, such as about 68.75 µg to about 181.25 µg, about 75 µg to about 175 µg, about 81.25 µg to about 168.75 µg, about 87.5 µg to about 162.5 µg, about 93.75 µg to about 156.25 µg, about 100 µg to about 150, about 106.25 µg to about 143.75 µg, about 112.5 µg to about 137.5 µg, or about 118.75 µg to about 131.25 µg.

In embodiments, for a target oral dose of 125 µg, 250 µg, or 500 µg, the loading dose amount may be in the range of about 60 µg to about 1000 µg, such as about 275 µg to about 750 µg, about 300 µg to about 725 µg, about 325 µg to about 700 µg, about 350 µg to about 650 µg, about 375 µg to about 625 µg, about 400 µg to about 600 µg, about 425 µg to about 575 µg, about 450 µg to about 550 µg, or about 475 µg to about 525 µg.

For example, in embodiments, for a target oral dose of 125 µg, 250 µg, or 500 µg, and for a patient with atrial fibrillation, atrial flutter, paroxysmal atrial fibrillation or ventricular fibrillation (who is in sinus rhythm or needs to be converted to sinus rhythm) the IV loading dose amount (which may be administered using one or more IV doses) may be in the range of up to about 1000 µg, such as about 100 µg to about 750 µg, about 175 µg to about 725 µg, about 200 µg to about 700 µg, about 300 µg to about 650 µg, about 375 µg to about 625 µg, about 400 µg to about 600 µg, about 425 µg to about 575 µg, about 450 µg to about 550 µg, or about 475 µg to about 525 µg, or any range within these endpoints.

Additionally, in embodiments, for a target oral dose of 125 µg, 250 µg, or 500 µg, and for a patient with ventricular tachycardia, atrial tachycardia, hemodynamically stable or unstable ventricular tachycardia, ventricular arrhythmia, premature ventricular contractions (PVCs), paroxysmal supraventricular tachycardia, supraventricular tachycardia, junctional ectopic tachycardia or junctional tachycardia (who is in sinus rhythm or needs to be converted to sinus rhythm) the IV loading dose amount (which may be administered using one or more IV doses) may be in the range of up to about 1000 µg, such as about 75 µg to about 800 µg, about 100 µg to about 750 µg, about 150 µg to about 700 µg, about 200 µg to about 650 µg, about 225 µg to about 625 µg, about 275 µg to about 600 µg, about 325 µg to about 575 µg, about 425 µg to about 550 µg, or about 450 µg to about 900 µg, or any range within these endpoints.

Further, for example, in embodiments, for a target oral dose of 125 µg, 250 µg, or 500 µg, and for a patient with heart failure, coronary artery disease or pulmonary artery hypertension (who is in sinus rhythm or needs to be converted to sinus rhythm) the IV loading dose amount (which may be administered using one or more IV doses) may be in the range of up to about 1000 µg, such as about 60 µg to about 750 µg, about 90 µg to about 775 µg, about 100 µg to about 900 µg, about 200 µg to about 550 µg, about 300 µg to about 625 µg, about 350 µg to about 600 µg, about 400 µg to about 575 µg, about 425 µg to about 650 µg, or about 475 µg to about 800 µg, or any range within these endpoints.

In embodiments, the IV loading dose is administered while the patient/subject is admitted to a hospital or other facility capable of providing cardiac resuscitation and continuous electrocardiographic monitoring. The loading dose can be slowly titrated upward until restoration of normal sinus rhythm as measured by electrocardiogram (ECG), or ceased or adjusted downward if ventricular arrhythmias, ventricular tachycardia, or other side effects of dofetilide, such as dizziness, are observed. In embodiments, the loading dose can be adjusted for patient QT interval or QTc as described below. In embodiments, the loading dose can be adjusted for patient creatinine clearance as described below.

In embodiments, the IV loading dose is delivered by way of an IV bolus or infusion over a period of about 60 minutes, such as about 0.5, 1, 2, 3, 4, 5, 6, 7, 8, 9, 10, 11, 12, 13, 14, 15, 20, 25, 30, 35, 40, 45, 50, 55, 60, 65, or 70 minutes, or within any range with any of these values as lower and upper values, such as 5 to 20 minutes, 10 to 40 minutes, 15 to 30 minutes, 20 to 45 minutes, 30 to 60 minutes, 35 to 50 minutes, 35 to 60 minutes, 40 to 60 minutes, 45 to 60 minutes, and so on.

The loading dose can be intravenously infused into a central or peripheral vein of the patient. The flow rate of the intravenous dose can be adjusted based on the concentration of the dofetilide in the intravenous formulation, the desired duration of administration, and the body weight of the patient to achieve a specific loading dose. Thus, longer administration durations of a specific dose (X µg/kg or mg/kg) will typically have slower flow rates compared to shorter administration durations to achieve the same dose.

According to some embodiments, after the loading dose, the dofetilide can be infused intravenously as a loading or maintenance dose at a rate in the range of 0.1 to 10 µg/kg/hr, including 0.1, 0.2, 0.3, 0.4, 0.5, 0.6, 0.7, 0.8, 0.9, 1.0, 1.2, 1.3, 1.4, 1.5, 1.6, 1.8, 1.9. 2.0, 2.2, 2.4, 2.5, 2.6, 2.7, 2.8, 3.0, 3.1, 3.2, 3.3, 3.4, 3.5, 3.6, 3.8, 4.0, 4.2, 4.3, 4.4, 4.5, 4.6, 4.7, 4.8, 5.0, 5.1, 5.2, 5.4, 5.5, 5.6, 5.7, 5.8, 6.0, 6.1, 6.2, 6.4, 6.5, 6.6, 6.8, 6.9, 7.0, 7.2, 7.4, 7.5, 7.6, 7.7, 7.8, 7.9, 8.0, 8.1, 8.2, 8.3, 8.4, 8.5, 8.6, 8.8, 8.9, 9.0, 9.2, 9.4, 9.5, 9.6, 9.7, 9.8, 9.9, 10.0 µg/kg/hr, or in any range with any of these values as lower or upper values, such as 1.2 to 2.8 µg/kg/hr,2.6 to 7.4 µg/kg/hr,3.4 to 5.8 µg/kg/hr, 4.2 to 8.6 µg/kg/hr, 1.0 to 3.0 µg/kg/hr,2.8 to 3.6 µg/kg/hr,1.0 to 4 µg/kg/hr, 1.2 to 4 µg/kg/hr, 1.4 to 4 µg/kg/hr, 1.6 to 4 µg/kg/hr, 1.8 to 4 µg/kg/hr, 2.0 to 4 µg/kg/hr,2.2 to 4 µg/kg/hr,2.4 to 4 µg/kg/hr,2.6 to 4 µg/kg/hr,2.7 to 4 µg/kg/hr,2.8 to 4 µg/kg/hr,1.0 to 5 µg/kg/hr,1.2 to 5 µg/kg/hr,1.4 to 5 µg/kg/hr,1.6 to 5 µg/kg/hr,1.8 to 5 µg/kg/hr,2.0 to 5 µg/kg/hr,2.2 to 5 µg/kg/hr,2.4 to 5 µg/kg/hr,2.6 to 5 µg/kg/hr,2.7 to 5 µg/kg/hr, 2.8 to 5 µg/kg/hr,3.0 to 5 µg/kg/hr,3.2 to 5 µg/kg/hr,3.4 to 5 µg/kg/hr,3.6 to 5 µg/kg/hr,1.0 to 6 µg/kg/hr,1.2 to 6 µg/kg/hr,1.4 to 6 µg/kg/hr,1.6 to 6 µg/kg/hr,1.8 to 6 µg/kg/hr,2.0 to 6 µg/kg/hr,2.2 to 6 µg/kg/hr,2.4 to 6 µg/kg/hr,2.6 to 6 µg/kg/hr,2.7 to 6 µg/kg/hr,2.8 to 6 µg/kg/hr,3.0 to 6 µg/kg/hr,3.2 to 6 µg/kg/hr,3.2 to 6 µg/kg/hr,3.4 to 6 µg/kg/hr, 3.6 to 6 µg/kg/hr,3.8 to 6 µg/kg/hr,4.0 to 6 µg/kg/hr,1.0 to 8 µg/kg/hr,1.2 to 8 µg/kg/hr, 1.4 to 8 µg/kg/hr, 1.6 to 8 µg/kg/hr,1.8 to 8 µg/kg/hr,2.0 to 8 µg/kg/hr,2.2 to 8 µg/kg/hr, 2.4 to 8 µg/kg/hr,2.6 to 8 µg/kg/hr,2.7 to 8 µg/kg/hr,2.8 to 8 µg/kg/hr, 3.0 to 8 µg/kg/hr, 3.2 to 8 µg/kg/hr,3.4 to 8 µg/kg/hr, 3.6 to 8 µg/kg/hr, 3.8 to 8 µg/kg/hr, 4.0 to 8 µg/kg/hr, 4.2 to 8 µg/kg/hr,4.4 to 8 µg/kg/hr,4.6 to 8 µg/kg/hr,4.8 to 8 µg/kg/hr, 5.0 to 8 µg/kg/hr, 1.0 to 10 µg/kg/hr, 1.2 to 10 µg/kg/hr,1.4 to 10 µg/kg/hr,1.6 to 10 µg/kg/hr,1.8 to 10 µg/kg/hr,2.0 to 10 µg/kg/hr,2.2 to 10 µg/kg/hr,2.4 to 10 µg/kg/hr,2.6 to 10 µg/kg/hr,2.7 to 10 µg/kg/hr,2.8 to 10 µg/kg/hr,3.0 to 10 µg/kg/hr,3.2 to 10 µg/kg/hr,3.4 to 10 µg/kg/hr, 3.6 to 10 µg/kg/hr,3.8 to 10 µg/kg/hr,4.0 to 10 µg/kg/hr,4.2 to 10 µg/kg/hr,4.4 to 10 µg/kg/hr,4.6 to 10 µg/kg/hr,4.8 to 10 µg/kg/hr,and 5.0 to 10 µg/kg/hr 5.2 to 10 µg/kg/hr,5.4 to 10 µg/kg/hr,5.6 to 10 µg/kg/hr,5.8 to 10 µg/kg/hr,6.0 to 10 µg/kg/hr,and so on. In embodiments, the maintenance dose is initiated at a higher rate and then titrated down. In other embodiments, the maintenance dose is initiated at a lower rate and then increased.

The maintenance dose (IV or oral) can be administered over a duration of up to about 60 minutes to several hours to several days to several months to several years, including 0.5, 1.0, 1.5, 2.0, 2.5, 3.0, 3.5, 4.0, 4.5, 5.0, 5.5, 6.0, 6.5, 7.0, 7.5, 8.0, 9, 10, 11, 12, 13, 14, 15, 16, 17, 18, 19, 20, 21, 22, 23, 24, 36, 48, 72 hours, including 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 days, or including 1, 2, 3, 4, 5, 6, 7, 8, 9, 10, 11, 12 months, or including 1, 2, 3, 4, 5, 8, or 10 years, or in any range with any of these values as lower and upper values, such as 0.5 to 1.5 hours, 1 to 2 hours, 1 to 3 hours, 1 to 4 hours, 1 to 5 hours, 1 to 6 hours, 1 to 10 hours, 1 to 18 hours, 1 to 24 hours, 1 to 36 hours, 1 to 48 hours, 1 to 72 hours, 1 to 168 hours, 2 to 3 hours, 2 to 4 hours, 2 to 5 hours, 2 to 6 hours, 2 to 8 hours, 2 to 10 hours, 2 to 18 hours, 2 to 24 hours, 2 to 36 hours, 2 to 48 hours, 2 to 72 hours, 2 to 168 hours, 3 to 4 hours, 3 to 5 hours, 3 to 6 hours, 3 to 10 hours, 3 to 18 hours, 3 to 24 hours, 3 to 36 hours, 3 to 48 hours, 3 to 72 hours, 3 to 168 hours, 4 to 8 hours, 4 to 10 hours, 4 to 18 hours, 4 to 24 hours, 4 to 36 hours, 4 to 48 hours, 4 to 72 hours, 4 to 168 hours, 6 to 8 hours, 6 to 10 hours, 6 to 18 hours, 6 to 24 hours, 6 to 36 hours, 6 to 48 hours, 6 to 72 hours, 6 to 168 hours, 8 to 10 hours, 8 to 18 hours, 8 to 24 hours, 8 to 36 hours, 8 to 48 hours, 8 to 72 hours, 8 to 168 hours, 12 to 24 hours, 12 to 72 hours, 12 to 168 hours, 24 to 72 hours, 1 to 7 days, 1 to 10 days, 1 to 14 days, 7 to 30 days, 1 to 3 months, 2 to 6 months, 1 to 5 years, and so on.

In embodiments, one or more IV or oral maintenance dose is administered, with a first maintenance dose being administered up to about 12 hours after the conclusion of the administration of the IV loading dose, such as immediately after the conclusion of the administration or up to about 6 hours after, such as about 0.5 hours, 0.75 hours, 1 hour, 1.25 hours, 1.5 hours, 1.75 hours, 2 hours, 2.25 hours, 2.5 hours, 2.75 hours, 3 hours, 3.25 hours, 3.5 hours, 3.75 hours, 4 hours, 4.25 hours, 4.5 hours, 4.75 hours, 5 hours, 5.25 hours, 5.5 hours, or 5.75 hours after administration of the IV loading dose. In embodiments, the first maintenance dose is administered about 1 hour to about 12 hours after conclusion of the administration of the loading dose, such as about 2 hours to about 11 hours, about 3 hours to about 10 hours, about 4 hours to about 9 hours, about 5 hours to about 8 hours, or about 6 hours to about 7 hours. In embodiments, the subject/patient is administered the first maintenance dose after being released from the hospital or other facility that was providing the monitoring, but the patient/subject can still be monitored in a similar way, such as by using a portable/wearable ECG monitoring system.

The maintenance dose can be intravenously infused into a central or peripheral vein of the patient. In other embodiments, the maintenance dose can be given orally. In an embodiment, the maintenance dose is initiated as an IV infusion, then transitioned to oral administration. In embodiments, multiple maintenance doses are given, wherein one or more maintenance dose(s) is administered as an IV infusion and one or more maintenance dose(s) is administered orally. In other embodiments, all maintenance doses are administered orally. In other embodiments, all maintenance doses are administered intravenously.

In embodiments, the one or more oral dose is given in an amount of about 500 µg, about 250 µg, or about 125 µg. In embodiments, more than one oral dose amount is given to a particular patient (for example, a patient receives one or more oral dose at 250 µg and a subsequent higher oral dose of 500 µg or a subsequent lower oral dose of 125 µg, or any combination thereof).

The patient can be monitored by electrocardiogram (ECG) for normal sinus rhythm and/or ventricular arrhythmias or ventricular tachycardia and the maintenance dose rate adjusted upward until normal sinus rhythm is reached or adjusted downward if ventricular arrhythmias, ventricular tachycardia, or other side effects typically associated with dofetilide administration, such as dizziness occur. The maintenance dose can be adjusted for patient QT or QTc interval as described below. The maintenance dose rate can be adjusted for patient creatinine clearance as described below.

The flow rate of the intravenous dose can be adjusted based on the concentration of dofetilide in the intravenous formulation and the body weight of the patient to achieve a specific maintenance dose rate in µg/kg/hr. The maintenance dose rate can be targeted to reach a particular therapeutic, steady-state plasma concentration based on established pharmacokinetic parameters of dofetilide such as half-life and clearance. As can be appreciated, the cumulative maintenance dose is a product of the rate of intravenous infusion in µg/kg/hr multiplied by the infusion duration in hours.

In embodiments, the IV loading dose is administered in a hospital or other facility that can provide cardiac resuscitation and continuous electrocardiographic monitoring and a first maintenance dose and subsequent maintenance doses are administered by the patient after discharge from the facility/hospital, but may still be under monitoring, such as self-monitoring. In other embodiments, the IV loading dose and a first maintenance dose are administered in a hospital or other facility that can provide cardiac resuscitation and continuous electrocardiographic monitoring and subsequent maintenance doses are administered by the patient after discharge from the facility/hospital.

In embodiments, subsequent maintenance doses are given after the first maintenance dose. In embodiments, the subsequent maintenance doses are administered at intervals of about 12 hours, about 24 hours, or about 48 hours. In embodiments, the subject/patient is administered subsequent maintenance dose(s) after being released from the hospital or other facility capable of providing cardiac resuscitation and continuous electrocardiographic monitoring.

In embodiments, the IV loading dose and/or maintenance dose(s) are given in amounts and at time intervals such that the patient achieves or is capable of achieving Cₘₐₓ ss within about 2 hours of the start of the administration of the IV loading dose. In embodiments, Cₘₐₓ ss is achieved before administration of the first maintenance dose.

In embodiments, the IV loading dose is given in an amount such that the patient experiences a Cₘₐₓ that is at least about 80% of the Cₘₐₓ ss for the patient’s specific dosing protocol within about 2 hours of initiation of the loading dose. In embodiments, following the IV loading dose, such as before the first oral dose, the patient experiences or is capable of experiencing a Cₘₐₓ that is at least about 85% of the Cₘₐₓ ss, such as about 87%, 90%, 92%, 95%, 97% or 99% of the Cₘₐₓ ss for the patient’s specific dosing protocol.

### Qt Interval Monitoring

Proper dosing for dofetilide can be ascertained by monitoring the QT interval, or the heart rate corrected QT (the QTc), during and/or after IV infusion of the loading and/or maintenance dose(s), and thus avoid the development of life-threatening ventricular tachycardias such as Torsades de Pointes. Patients can be monitored by electrocardiogram (ECG) and a baseline QT or QTc can be measured. A baseline QT or QTc exceeding a certain threshold, such as 440 msec (or 500 msec for patients with ventricular conduction abnormalities), may exclude some patients from dofetilide treatment (see U.S. Pat. Application Publication No. 20190388371A1). For patients that qualify for treatment, the QT and/or QTc can be monitored during infusion of the loading and/or maintenance dose, and such dose can be adjusted downward or ceased if the patient’s QT or QTc becomes too high, either over a QT or QTc threshold or a change exceeding a patient’s baseline QT or QTc. In one implementation, the QT or QTc threshold is 500 msec. In various implementations, the change exceeding baseline QTc can be 5%, 10%, 15%, or 20% over baseline QT or QTc. The QT or QTc can be measured at regular intervals, such as every 10, 15, 20, 30, 45, or 60 minutes during treatment. In some situations, such as if the patient’s heart rate is less than 60 bpm, the uncorrected QT interval can be used. Remote ECG monitoring, such as Holter monitoring or event monitoring, may be indicated for long term monitoring of QT or QTc interval. In embodiments, the patient/subject is not monitored using a portable/wearable ECG monitoring system after release from the hospital or other facility that was providing monitoring.

In embodiments, one or more of the maintenance doses is selected based on a patient’s change in QT or QTc (for example, a patient that experiences an increase in QTc of less than 20% of baseline after receiving the loading dose may receive a higher maintenance dose than a patient that experiences an increase in QT or QTc of 15% or greater after receiving the loading dose). In embodiments, the maintenance dose is changed after administration of a previous maintenance dose due to a change in the patient’s QTc (for example, a patient given an oral dose of 250 µg experiences an increase in QTc of 15% or greater, and the next oral dose can be lowered to 125 µg).

### Creatinine Clearance

Because elimination of dofetilide is primarily renal, renal impairment can potentially lead to toxic serum concentrations. As such, it is desirable to measure patient creatinine clearance prior to initiating a dofetilide dosing regimen in order to gauge kidney function. Serum samples can be taken from the patient prior to treatment with dofetilide and serum creatinine measured according the following formulas:

$$\begin{matrix}\text{Creatinine clearance} \left(\text{male}\right) = \\ \left[\left(140 - \text{Age}\right) \times \text{Body Wt} \left(\text{kg}\right)\right] / \left[72 \times \text{serum creainine}\right] \left(\text{mg} / \text{dL}\right) ]\end{matrix}$$

$$\text{Creatinine clearence} \left(\text{female}\right) = \text{Creatine clearance} \left(\text{male}\right) \times 0.85$$

The loading dose and maintenance dose can be adjusted according to creatinine clearance as provided in Table I.

TABLE I

| Creatinine Clearance (mL/min) | Loading Dose | Maintenance Dose |
|---|---|---|
| >60 | High | High |
| 40 - 60 | Medium | Medium |
| 20 - <40 | Low | Low |
| <20 | Dofetilide not indicated | Dofetilide not indicated |

In other embodiments, a patient/subject with a lower creatinine clearance may receive a lower loading dose or maintenance dose than a patient with a higher creatinine clearance, or in some cases a patient/subject with a higher creatinine clearance may receive a lower loading dose or maintenance dose than a patient with a lower creatinine clearance. In some cases, for example, the amount of dofetilide, whether by IV or an oral dose, appropriate to administer to a subject with a CrCl of 20 - <40 mL/min or 40-60 mL/min can be higher than is appropriate for a subject with a CrCl of >60 mL/min. Even further, in certain situations dofetilide may be administered to a patient with a creatinine clearance of <20 mL/min, such as at a fraction of the dose that would be used for patients with normal kidney function, and/or at longer dosing intervals.

### Indications

In embodiments, dofetilide is administered intravenously according to any of the above loading and/or maintenance doses and durations to treat or prevent such conditions as atrial fibrillation or flutter (sustained or intermittent), paroxysmal atrial fibrillation, ventricular tachycardia, ventricular fibrillation, paroxysmal supraventricular tachycardia, heart failure, coronary artery disease, pulmonary artery hypertension, and the like, and for situations where the patient is unable to take an anti-arrhythmic by mouth (NPO). The specific intravenous loading dose and intravenous maintenance dose rate protocols would be selected based upon the patient’s condition, baseline and in-treatment QT interval or QTc, as well as the patient’s creatinine clearance as described herein. Other factors affecting selection of the loading and maintenance dose include patient body weight. Oral maintenance doses of dofetilide can be administered to the patient according to available oral dosages (125 µg, 250 µg, 500 µg, typically given twice daily), QTc, creatinine clearance, body weight, and other factors, as described by U.S. Pat. Application Publication No. 20190388371A1). Situations where IV loading and/or maintenance doses are appropriate include those in which the patient cannot take oral administration (NPO), or for gastrointestinal conditions resulting in poor absorption, recovery from GI surgery, and/or intensive care.

Dofetilide administration according to embodiments of the invention is administered to patients naïve to dofetilide treatment and/or to patients currently receiving dofetilide therapy and/or to patients having previously received dofetilide.

In embodiments, the dofetilide dose (including the loading dose and/or maintenance dose, whether IV or oral) is determined by patient characteristics including body weight, sex, and/or creatinine clearance.

The following Examples are provided to illustrate various protocols for administering dofetilide and are applicable to subjects with a cardiovascular condition selected from atrial fibrillation, atrial flutter, atrial tachycardia, ventricular tachycardia, hemodynamically stable or unstable ventricular tachycardia, paroxysmal atrial fibrillation, ventricular fibrillation, ventricular arrhythmia, premature ventricular contractions (PVCs), paroxysmal supraventricular tachycardia, supraventricular tachycardia, junctional ectopic tachycardia, junctional tachycardia, heart failure, coronary artery disease, and pulmonary artery hypertension. The Examples are intended as examples only and are intended to be adapted according to other portions of this disclosure and based on a particular patient’s needs.

### Example 1

An example dofetilide treatment protocol for a patient is described herein. The male patient is admitted to the hospital with a cardiovascular condition. The patient can be admitted to any facility capable of providing cardiac resuscitation and continuous electrocardiographic monitoring. The patient can be currently in sinus rhythm, recently cardioverted to sinus rhythm, or administered dofetilide to convert the patient to sinus rhythm. The patient’s creatinine clearance is measured/determined. The patient is connected to an electrocardiograph, a baseline QT interval (or QTc) is measured/determined, and treatment is initiated with an IV loading dose of dofetilide in an amount of up to about 750 µg infused over about 1 hour. The amount of the dofetilide IV dose is based on the target oral dose and the creatinine clearance of the patient, such as an amount of dofetilide in the range of 400-450 µg (for a CrCl of >60 mL/min), 425-550 µg (for a CrCl in the range of 40-60 mL/min), or 440-475 µg (for a CrCl in the range of 20 to <40 mL/min) for a target oral dose of 500 µg.

The patient’s QT interval is measured/determined prior to start of the IV (baseline) and is monitored every 15 minutes or so during the IV loading dose administration, optionally along with heart rate (HR) and blood pressure (BP). The patient’s QTc is calculated from the QT measurement. If the patient’s baseline (i.e., prior to dofetilide administration) QT interval or baseline QTc is greater than 440 msec (or 500 msec in patients with ventricular conduction abnormalities), then dofetilide is contraindicated. During the IV loading dose, if the QT interval or QTc increases by greater than 15% of the patient’s baseline QT interval or baseline QTc (or is greater than 500 msec or 550 msec in patients with ventricular conduction abnormalities), then the IV dofetilide is discontinued or a subsequent lower dose is considered or administered. If after any subsequent IV or oral dose the QT or QTc increases to greater than 500 msec (or 550 msec in patients with ventricular conduction abnormalities) or increases by greater than 15% of the patient’s baseline QT or baseline QTc, then dofetilide is discontinued.

Once it is determined that the patient/subject is capable of tolerating the dofetilide by way of the IV loading dose, oral dosing begins after the patient/subject has been released from the hospital or other facility that was providing the monitoring. The patient/subject can still be monitored in a similar way, such as by using a portable/wearable ECG monitoring system. In other embodiments, the patient/subject is not monitored using a portable/wearable ECG monitoring system after release from the hospital or other facility that was providing monitoring. Up to 12 hours after completion of the loading dose, the patient receives an oral maintenance dose of 500 µg of dofetilide. The patient’s QT interval or QTc is optionally monitored every hour after the administration of the oral maintenance dose, such as by way of a portable/wearable ECG monitoring system. If after the oral maintenance dose the patient’s QT or QTc is greater than 500 msec or (550 msec in patients with ventricular conduction abnormalities) or if the QT or QTc increases by greater than 15% of the patient’s baseline QT or QTc, then the next oral maintenance dose given is reduced to 250 µg of dofetilide and/or is administered 24 to 48 hours after the last oral dose.

For the situation where QT interval or QTc is within an acceptable range, oral B.I.D. dofetilide treatment is continued. For the situation where (i) the patient’s QT or QTc after administration of the first oral maintenance dose is greater than 550 msec, (ii) the patient’s subsequent dose is reduced, and (iii) the QT interval or QTc for the subsequent reduced maintenance dose (or any subsequent maintenance dose) is observed to be greater than 500 msec (or 550 msec in patients with ventricular conduction abnormalities) or increases by greater than 15% of the patient’s baseline QT or QTc, additional subsequent oral maintenance doses can be further reduced to 125 µg, and/or dofetilide treatment is discontinued.

### Example 2

A patient with a cardiovascular condition and in need of dofetilide treatment is admitted to the hospital or other facility capable of providing cardiac resuscitation and continuous electrocardiographic monitoring. Treatment is initiated for a dofetilide dosing protocol of 250 µg BID with an IV loading dose of up to 375 µg infused over up to 1 hour. The amount of the dofetilide IV dose is based on the creatinine clearance of the patient, such as an amount of dofetilide in the range of 175-200 µg (for a CrC1 of >60 mL/min), 190-300 µg (for a CrC1 in the range of 40-60 mL/min), or 210-240 µg (for a CrCl in the range of 20 to <40 mL/min). The loading dose may be administered by way of two or several smaller IV doses. The patient’s QT or QTc interval is measured/determined every 15 minutes or so during the IV loading dose administration along with HR and BP.

During or following administration of the IV loading dose, the patient’s QT or QTc interval is determined not to be greater than 500 msec (or 550 msec for patients with ventricular conduction abnormalities) or does not increase by greater than 15% of the patient’s baseline QT or QTc.

The patient is discharged with instructions to self-administer an oral maintenance dose in an amount of 250 µg within 12 hours of the completion of the IV loading dose. The patient is discharged with a portable/wearable ECG monitoring system. After self-administration of the oral maintenance dose, the patient’s QTc interval is determined to be greater than 500 msec (or 550 msec for patients with ventricular conduction abnormalities) or increases by greater than 15% of the patient’s baseline QT or QTc. The patient can be re-admitted to the hospital. A reduced maintenance dose of 125 µg is administered to the patient orally or by IV. After administration of the reduced maintenance dose, the patient’s QT or QTc interval is determined to not be greater than 500 msec (or 550 msec for patients with ventricular conduction abnormalities) and the ΔQT or ΔQTc is not greater than 15% of the patient’s initial QT or QTc.

If admitted to the hospital after being administered an oral dose, the patient is discharged with instructions to orally self-administer subsequent maintenance doses of 125 µg every 12 to 48 hours. The patient can optionally still be monitored using a portable/wearable ECG monitoring system.

### Example 3

An example anti-arrhythmic/dofetilide treatment protocol is described herein. The patient has a cardiovascular condition and is admitted to the hospital or other facility capable of providing cardiac resuscitation and continuous electrocardiographic monitoring. The patient’s creatinine clearance is determined to be between 20 and <40 mL/min. The patient is connected to an electrocardiograph, and an initial QT or QTc interval is determined to be not greater than 440 msec (or 500 msec in patients with ventricular conduction abnormalities). Treatment is initiated with an IV loading dose of up to 12 µg/kg infused over a period of up to 1 hour, such as in the range of about 105-115 µg (for a target maintenance dose of 125 µg), 210-240 µg (for a target maintenance dose of 250 µg), or 440-475 µg (for a target maintenance dose of 500 µg). The loading dose may be administered by way of two or more smaller IV doses. The patient’s QT or QTc interval is measured/determined every 15 minutes or so during the IV loading dose administration along with HR and BP.

During and following administration of the loading dose, the patient’s QT or QTc interval is determined to be not greater than 500 msec (or 550 msec for patients with ventricular conduction abnormalities) and ΔQT or ΔQTc is not greater than 15% of the patient’s initial QT or QTc. After the IV dose, the patient is discharged from the hospital/facility with instructions to administer oral maintenance doses of 125 µg, 250 µg, or 500 µg, accordingly, within 12 hours of completion of the IV loading dose. The patient can optionally self-monitor QT or QTc interval following administration of the oral maintenance dose, such as by using a portable/wearable ECG monitoring system. The patient’s QT or QTc interval still does not exceed 500 msec (or 550 msec for patients with ventricular conduction abnormalities) and is not greater than 15% of the patient’s initial QT or QTc. The patient would be instructed to self-administer subsequent oral maintenance doses of 125 µg, 250 µg, or 500 µg, accordingly, at a 12 to 48 hour interval from the first oral dose.

### Example 4

An example dofetilide treatment is described herein. The patient is admitted to the hospital or other facility capable of providing cardiac resuscitation and continuous electrocardiographic monitoring to initiate treatment for a cardiovascular condition. The patient’s creatinine clearance is determined to be in the range of 40-60 mL/min. The patient is connected to an electrocardiograph, and an initial QT or QTc interval is measured/determined. Treatment is initiated with an IV loading dose based on an oral target dose of 500 µg, which IV dose is administered in an amount in the range of ± 50% of the oral target dose, such as an amount within the range of from about 250-750 µg, and can be adjusted based on the creatinine clearance of the patient. In this example, the IV dose can be in the range of 425-550 µg (for a CrCl of 40-60 mL/min), and is infused over a period of up to 1 hour. The patient’s QT or QTc interval is measured/determined every 15 minutes or so during the IV loading dose administration along with HR and BP.

During and following administration of the loading dose, the patient’s QT or QTc interval is determined to be not greater than 500 msec (or 550 msec for patients with ventricular conduction abnormalities) and the ΔQT or ΔQTc is not greater than 15% of the patient’s initial QT or QTc. The patient would be discharged with instructions to self-administer an oral maintenance dose in an amount of 500 µg within 12 hours of the completion of the IV loading dose. The patient’s QT interval may be monitored following administration of the oral maintenance dose, such as by way of a portable/wearable ECG monitoring system. The patient would be expected to experience a Cₘₐₓ ss within 2 hours of the initiation of the IV loading dose. The patient’s QT or QTc interval still does not exceed 500 msec (or 550 msec for patients with ventricular conduction abnormalities) and the ΔQT or ΔQTc is not greater than 15% of the patient’s initial QT or QTc. The patient would be instructed to self-administer subsequent oral maintenance doses of 500 µg at a 12-hour interval from the first oral dose.

### Example 5

An example anti-arrhythmic/dofetilide treatment protocol is described herein for a patient with a cardiovascular condition. The female patient is admitted to the hospital or other facility capable of providing cardiac resuscitation and continuous electrocardiographic monitoring to initiate treatment. The patient’s creatinine clearance is determined to be between 40 and 60 mL/min. The patient is connected to an electrocardiograph, and an initial QT or QTc interval is determined. Treatment is initiated with an IV loading dose based on an oral target of 250 µg, which is administered in an amount in the range of ± 50% of the oral target dose, such as in an amount in the range of from about 190-300 µg, and is infused over a period of up to about 1 hour. The patient’s QT or QTc interval is measured/determined during the IV loading dose administration, optionally every 15 minutes or so, along with HR and BP.

During and following administration of the loading dose, the patient’s QT or QTc interval is determined to be not greater than 500 msec (or 550 msec in patients with ventricular conduction abnormalities) and the ΔQT or ΔQTc is not greater than 15% of the patient’s initial QT or QTc. The patient is discharged from the hospital with instructions to be administered subsequent IV and/or oral maintenance doses of up to 250 µg, optionally every 12 to 48 hours. A maintenance dose is administered to the patient as an IV (such as a bolus dose), or oral dose of 250 µg. The patient’s QT or QTc interval is monitored during and following administration of the IV or oral maintenance dose. The patient’s QT or QTc interval still does not exceed 500 msec (or 550 msec in patients with ventricular conduction abnormalities) and the ΔQT or ΔQTc is not greater than 15% of the patient’s initial QT or QTc. Maintenance dose administration can be continued for any desired period of time.

### Example 6

An example dofetilide treatment protocol for a patient is described herein. A patient with ventricular arrhythmia is admitted to a hospital (or any facility capable of providing cardiac resuscitation and continuous electrocardiographic monitoring). The patient’s creatinine clearance is measured/determined. The patient is connected to an electrocardiograph, a baseline QT interval (or QTc) is measured/determined and the patient is determined to be in an acceptable range (i.e., having a QT or QTc interval as described in this disclosure). The patient is administered dofetilide to convert the patient to sinus rhythm, or the patient can already be in sinus rhythm or recently converted to sinus rhythm with dofetilide or another antiarrhythmic drug, or other cardioversion technique. Treatment is initiated for a target oral dose of 500 µg B.I.D., starting with an IV loading dose of dofetilide in an amount of up to about 1000 µg infused over about 1 hour. The amount of the dofetilide IV dose is based on the target oral dose and the creatinine clearance of the patient as provided in this disclosure for cardiovascular conditions. Once it is determined that the patient/subject is in sinus rhythm (or is maintaining sinus rhythm) and is capable of tolerating the dofetilide by way of the IV loading dose, oral dosing begins after the patient/subject has been released from the hospital or other facility that was providing the monitoring. The patient/subject upon release from the monitoring facility may still be monitored in a similar way, or not. Up to 12 hours after completion of the loading dose, the patient receives an oral maintenance dose of 500 µg of dofetilide and continues the oral dosing B.I.D. or once a day, so long as the patient tolerates the drug, e.g., the patient maintains an acceptable QT or QTc interval and/or continues to be in sinus rhythm and/or does not experience ventricular arrhythmia or another cardiovascular condition.

### Example 7

An example dofetilide treatment protocol for a patient with atrial fibrillation and/or atrial flutter is described herein. The patient is admitted to a hospital or any facility capable of providing cardiac resuscitation and continuous electrocardiographic monitoring. The patient is connected to an electrocardiograph, a baseline QT interval (or QTc) is measured/determined and the patient is determined to be in an acceptable range (i.e., having a QT or QTc interval as described in this disclosure). The patient’s creatinine clearance is measured/determined. The patient is administered dofetilide to convert the patient to sinus rhythm, or the patient can already be in sinus rhythm or recently converted to sinus rhythm with dofetilide or another antiarrhythmic drug, or other cardioversion technique. Treatment is initiated for a target oral dose of 250 µg B .I.D., starting with an IV loading dose of dofetilide in an amount of up to about 750 µg infused over about 1 hour. The amount of the dofetilide IV dose is based on the target oral dose and the creatinine clearance of the patient as provided in this disclosure for cardiovascular conditions. Once it is determined that the patient/subject is in sinus rhythm (or is maintaining sinus rhythm) and is capable of tolerating the dofetilide by way of the IV loading dose, oral dosing begins after the patient/subject has been released from the hospital or other facility that was providing the monitoring. The patient/subject upon release from the monitoring facility may still be monitored in a similar way, or not. Up to 12 hours after completion of the loading dose, the patient receives an oral maintenance dose of 250 µg of dofetilide and continues the oral dosing B.I.D., so long as the patient tolerates the drug, e.g., the patient maintains an acceptable QT or QTc interval and/or continues to be in sinus rhythm and/or does not experience atrial fibrillation and/or atrial flutter or other cardiovascular condition, such as ventricular arrhythmia.

The present disclosure has described particular implementations having various features. In light of the disclosure provided herein, it will be apparent to those skilled in the art that various modifications and variations can be made without departing from the scope or spirit of the disclosure. One skilled in the art will recognize that the disclosed features may be used singularly, in any combination, or omitted based on the requirements and specifications of a given application or design. When an implementation refers to “comprising” certain features, it is to be understood that the implementations can alternatively “consist of” or “consist essentially of” any one or more of the features. Other implementations will be apparent to those skilled in the art from consideration of the specification and practice of the disclosure.

It is noted in particular that where a range of values is provided in this specification, each value between the upper and lower limits of that range is also specifically disclosed. The upper and lower limits of these smaller ranges may independently be included or excluded in the range as well. The singular forms “a,” “an,” and “the” include plural referents unless the context clearly dictates otherwise. It is intended that the specification and examples be considered as exemplary in nature and that variations that do not depart from the essence of the disclosure fall within the scope of the disclosure. Further, all of the references cited in this disclosure including patents, published applications, and non-patent literature are each individually incorporated by reference herein in their entireties and as such are intended to provide an efficient way of supplementing the enabling disclosure as well as provide background detailing the level of ordinary skill in the art.

1. A method of intravenously administering dofetilide, comprising:
administering an intravenous (IV) dosage of dofetilide to a subject over a period of about 1 hour in the range of ± 50% of a target oral dose;
wherein the IV dosage is administered while the subject is in a facility capable of providing cardiac resuscitation and electrocardiographic monitoring.

2. A method of administering dofetilide, comprising:
administering an intravenous (IV) dosage of dofetilide to a subject in an amount of about 62.5-1000 µg;
wherein the IV dosage is administered while the subject is in a facility capable of providing cardiac resuscitation and electrocardiographic monitoring; and
after administering the IV dosage, and after discharging the subject from the facility capable of providing cardiac resuscitation and electrocardiographic monitoring, administering an oral dosage of dofetilide to the subject in an amount of 125 µg, 250 µg or 500 µg.

3. The method of claim 1, further comprising administering and optionally repeating oral administration of dofetilide, wherein the administering or repeating of the oral administration is performed after the subject is discharged from the facility capable of providing cardiac resuscitation and electrocardiographic monitoring.

4. The method of claim 1, wherein the subject has a cardiovascular condition selected from atrial fibrillation, atrial flutter, atrial tachycardia, ventricular tachycardia, hemodynamically stable or unstable ventricular tachycardia, paroxysmal atrial fibrillation, ventricular fibrillation, ventricular arrhythmia, premature ventricular contractions (PVCs), paroxysmal supraventricular tachycardia, supraventricular tachycardia, junctional ectopic tachycardia, junctional tachycardia, heart failure, coronary artery disease, and pulmonary artery hypertension.

5. The method of claim 4, wherein the subject is in sinus rhythm and/or has been converted to sinus rhythm.

6. The method of claim 2, wherein:
the IV dosage is administered over a period of about 1 hour and:
the IV dosage comprises about 62.5-187.5 µg dofetilide and the oral dosage is 125 µg;
the IV dosage comprises about 125-375 µg dofetilide and the oral dosage is 250 µg; or
the IV dosage comprises about 250-1000 µg dofetilide and the oral dosage is 500 µg.

7. A method of administering dofetilide, comprising:
(A) before administering an IV dosage of dofetilide to a subject, determining a creatinine clearance (CrCl) of the subject, and selecting an amount of dofetilide to administer to the subject based on the creatinine clearance; and
(B) administering to the subject the IV dosage comprising the selected amount of dofetilide based on the creatinine clearance.

8. The method of claim 7, wherein the IV dosage is administered to the subject in a facility capable of providing cardiac resuscitation and continuous electrocardiographic monitoring.

9. The method of claim 8, wherein the IV dosage is based on a target oral dose of dofetilide and one or more oral dosages of dofetilide are administered to the subject out of the facility capable of providing cardiac resuscitation and continuous electrocardiographic monitoring.

10. The method of claim 7, further comprising administering one or more IV maintenance dose of dofetilide after administering the IV dosage of dofetilide.

11. The method of claim 7, further comprising administering one or more oral dose of dofetilide before or after administering the IV dosage of dofetilide.

12. The method of claim 7, wherein:
the IV dosage is administered over a period in the range of 1-5 hours.

13. The method of claim 7, wherein:
the IV dosage comprising the selected amount of dofetilide based on the creatinine clearance of the subject comprises an amount of dofetilide as follows:

| Maintenance Dose | Intravenous Dosage of Dofetilide | | |
|---|---|---|---|
| | Subject’s CrCl >60 mL/min | Subject’s CrCl 40-60 mL/min | Subject’s CrCl 20 to <40 mL/min |
| 125 µg | 50-150 µg | 100-250 µg | up to 125 µg |
| 250 µg | 100-350 µg | 200-500 µg | up to 250 µg |
| 500 µg | 300-800 µg | 400-1000 µg | up to 500 µg |

.

14. The method of claim 7, wherein the subject is capable of experiencing a dofetilide Cₘₐₓ steady state within about 2 hours of administration of the IV dosage.

15. The method of claim 7, wherein the IV dosage of dofetilide is administered:
in a first higher amount for the subject with a CrCl of 20 mL/min to <40 mL/min; and
in a second lower amount for the subject with a CrCl of 40 mL/min or greater.

16. The method of claim 1, wherein the subject is discharged with instructions or a prescription to administer one or more oral doses of dofetilide at a 12 to 48 hour interval for a selected period of time.

17. The method of claim 11, wherein the amount of dofetilide in one or more of the oral doses is different than the amount of dofetilide in one or more other of the oral doses.

18. The method of claim 1, wherein the IV dosage is administered to the subject in an amount and over a period of time such that the dofetilide hydrochloride reaches or is capable of reaching a Cₘₐₓ in the subject that is at least about 70% of a steady state Cₘₐₓ for an oral dosing protocol of 125 µg, 250 µg, or 500 µg dofetilide.

19. The method of claim 1, further comprising:
obtaining a QT or QTc interval of the subject before administration of the IV dosage; and
obtaining a QT or QTc interval of the subject during or after administration of the IV dosage, but before administration of any oral dosage;
wherein the QT or QTc interval during or after administering the IV dosage is less than a 15% increase from the QT or QTc interval before administering the IV dosage.

20. The method of claim 4, wherein the subject is not in sinus rhythm.
